(19) United States
(12) Patent Application Publication
(10) Pub. No.: US 2007/0191935 A1
(43) Pub. Date: Aug. 16, 2007

(54) Drug Delivery Stent with Extended In Vivo Drug Release

(75) Inventors: Theodore L. Parker, Danville, CA (US); John F. Shanley, Redwood City, CA (US); Frank Litvack, Los Angeles, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/671,642

(22) Filed: Feb. 6, 2007

Related U.S. Application Data
(60) Provisional application No. 60/765,702, filed on Feb. 6, 2006.

(51) Int. Cl. A61F 2/06

(52) U.S. Cl. 623/1.42; 623/1.15

(57) ABSTRACT

A method for reducing the level of restenosis following a stent placement medical intervention involves the continuous administration of a dose of an anti-restenotic agent, such as paclitaxel, from the stent to vascular tissue in need of treatment in a controlled and extended drug release profile for a period of at least 60 days in vivo. The in vivo release profile is determined by in vivo animal experiments involving implanting a series of stents in animals, explanting the stents from the animals at selected time points, and extracting remaining drug from the explanted stents.

### RELATED APPLICATIONS

This application claims priority to U.S. Provisional Patent Application Ser. No. 60/765,702, filed Feb. 6, 2006, the entire contents of which are incorporated herein by reference.

### BACKGROUND

Most coronary artery-related deaths are caused by atherosclerotic lesions which limit or obstruct coronary blood flow to heart tissue. To address coronary artery disease, doctors often resort to percutaneous transluminal coronary angioplasty (PTCA) or coronary artery bypass graft (CABG). PTCA is a procedure in which a small balloon catheter is passed down a narrowed coronary artery and then expanded to re-open the artery. The major advantage of angioplasty is that patients in which the procedure is successful need not undergo the more invasive surgical procedure of coronary artery bypass graft. A major difficulty with PTCA is the problem of post-angioplasty closure of the vessel, both immediately after PTCA (acute reocclusion) and in the long term (restenosis).

Coronary stents are typically used in combination with PTCA to reduce reocclusion of the artery. Stents are introduced percutaneously, and transported transluminally until positioned at a desired location. The stents are then expanded either mechanically, such as by the expansion of a mandrel or balloon positioned inside the stent, or expand themselves by releasing stored energy upon actuation within the body. Once expanded within the lumen stents become encapsulated within the body tissue and remain a permanent implant.

Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen. To treat restenosis, additional revascularization procedures are frequently required, thereby increasing trauma and risk to the patient.

While the exact mechanisms of restenosis are still being determined, certain agents have been demonstrated to reduce restenosis in humans. Drug eluting stents represent the most advanced and sophisticated treatment currently available to address restenosis. Two examples of agents which have been demonstrated to reduce restenosis when delivered from a stent are paclitaxel, a well-known compound that is commonly used in the treatment of cancerous tumors, and Rapamycin, an immunosuppressive compound used to prevent rejection of organ or tissue transplants.

Currently marketed drug-eluting stents are bare metal stents that are coated on the surface with a drug and a biostable polymer to reduce restenosis by inhibiting the growth or proliferation of neointima. In addition to polymer coated stents other polymer and non-polymer drug delivery systems are in development to allow delivery of antiproliferative drugs from stents.

Drug eluting stent systems are tested in various in vitro test systems to determine the kinetic release profile, also called the release kinetics, or amount of drug released from the polymer system over time. Clinical trials have demonstrated that a drug's release kinetics in addition to total dose have an effect on clinical outcomes. The in vitro test processes generally include placing a stent into an artificial release medium for a period of time, removing the stent from the release medium, and analyzing the release medium, such as by HPLC, to determine the amount of drug released from the stent during that period. This procedure is repeated at a number of time points and the cumulative drug release is plotted vs. time as a release kinetic profile. It has been shown that the release kinetic from the in vitro analysis can vary significantly depending on the release medium and test procedure used. Further it is difficult to compare different polymer/drug systems in an in vitro model since different polymers and drugs respond differently to the same release medium. In vitro release kinetics are seldom reflective of the in vivo release within an actual artery.

Thus, it would be desirable be able to characterize a release kinetic of a drug eluting stent based on in vivo data in an animal model which provides a close correlation to the human body.

### SUMMARY OF THE INVENTION

The present invention relates to methods of reducing restenosis and stents for reducing restenosis which deliver drug in vivo over an extended administration period of at least 60 days.

In accordance with one aspect of the invention, a method of reducing restenosis is comprised of providing a drug delivery stent having a dosage of paclitaxel for delivery to an artery, the dosage arranged such that substantially all the paclitaxel is releasable from the stent upon implantation of the stent in the artery, implanting the stent within an artery of a patient, and delivering paclitaxel from the stent in vivo over an administration period beginning on the date of implantation and ending between 60 days and 8 months after implantation, wherein after the administration period no paclitaxel remains on the stent.

In accordance with a further aspect of the invention, a method of reducing restenosis comprises the steps of providing a drug delivery stent having a dosage of antirestenotic drug for delivery to an artery, the dosage arranged such that substantially all the paclitaxel is releasable from the stent upon implantation of the stent in the artery, implanting the stent within an artery of a patient, and delivering drug from the stent in vivo over an administration period beginning on the date of implantation and ending between 60 days and 8 months after implantation, wherein after the administration period no drug remains on the stent.

In accordance with another aspect of the invention, a stent for reducing restenosis is comprised of a drug delivery stent having initial unexpanded diameter for insertion of the stent into a coronary artery and an expanded diameter for implantation within a coronary artery, the stent having a dosage of paclitaxel for delivery to an artery, the dosage arranged such that substantially all the paclitaxel is releasable from the stent upon implantation of the stent in the artery, wherein the dosage of paclitaxel is arranged to be released over an administration period beginning on the date of implantation and ending between 60 days and 8 month after implantation, wherein after the administration period no drug remains on the stent.

In accordance with an additional aspect of the invention, a method of reducing restenosis is comprised of providing a drug delivery stent having a dosage of antirestenotic drug for delivery to an artery, implanting the stent within an artery of a patient, and delivering drug from the stent in vivo over an administration period beginning on the date of implantation and ending within 6 months after implantation, wherein not more than 40% of the drug is delivered in the first 30 days and after the administration period no drug remains on the stent.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1.

FIG. 3 is a side cross sectional view of an example of an opening in a stent showing a matrix with a therapeutic agent and polymer.

FIG. 4 is a graph of the in vivo cumulative release and release rate of paclitaxel from a paclitaxel loaded stent system.

FIG. 5 is a graph of the in vivo release by percentage released of paclitaxel and polymer from a paclitaxel loaded stent system.

### DETAILED DESCRIPTION

A method for decreasing the level of restenosis following a stent placement medical intervention involves the continuous administration of a dose of an anti-restenotic agent or drug from the stent to vascular tissue in need of treatment in a controlled and extended in vivo drug release profile. It is envisioned that the vascular tissue in need of treatment is arterial tissue, specifically coronary arterial tissue. The method of extended in vivo release increases the therapeutic effectiveness of administration of a given dose of anti-restenotic agent and reduces side effects.

In one example described in detail herein the agent or drug will be contained in reservoirs in the stent body prior to release. In the reservoir example, the drug will be held within the reservoirs in the stent in a drug delivery matrix comprised of the drug and a polymeric material and optionally additives to regulate the drug release. Preferably the polymeric material is a bioresorbable polymer. Although a reservoir example is described, the drug delivery stent of the present invention can include matrices fixed to a stent in a variety of manners including reservoirs, coatings, microspheres, affixed with adhesion materials or combinations thereof,

The following terms, as used herein, shall have the following meanings:

The terms “drug” and “therapeutic agent” are used interchangeably to refer to any therapeutically active substance that is delivered to a living being to produce a desired, usually beneficial, effect.

The tern “matrix” or “biocompatible matrix” are used interchangeably to refer to a medium or material that, upon implantation in a subject, does not elicit a detrimental response sufficient to result in the rejection of the matrix. The matrix may contain or surround a therapeutic agent, and/or modulate the release of the therapeutic agent into the body. A matrix is also a medium that may simply provide support, structural integrity or structural barriers. The matrix may be polymeric, non-polymeric, hydrophobic, hydrophilic, lipophilic, amphiphilic, and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or exeretable, over a period of time from minutes to years, usually less than one year, while maintaining any requisite structural integrity in that same time period.

The term “openings” includes both through openings and recesses.

The term “pharmaceutically acceptable” refers to the characteristic of being non-toxic to a host or patient and suitable for maintaining the stability of a therapeutic agent and allowing the delivery of the therapeutic agent to target cells or tissue.

The term “polymer” refers to molecules formed from the chemical union of two or more repeating units, called monomers. Accordingly, included within the term “polymer” may be, for example, dimers, trimers, oligomers, and copolymers prepared from two or more different monomers. The polymer may be synthetic, naturally occurring or semisynthetic. In preferred form, the term “polymer” refers to molecules which typically have a Mw greater than about 3000 and preferably greater than about 10,000 and a Mw that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly(block-ethylene oxide-block-lactide-co-glycolide)polymers (PEO-block-PLGA and PEO-block-PLGA-block-PEO); polyethylene glycol and polyethylene oxide, poly(block-ethylene oxide-block-propylene oxide-block-ethylene oxide); polyvinyl pyrrolidone; polyorthoesters; polysaccharides and polysaccharide derivatives such as polyhyaluronic acid, poly(glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutainic acid, albumin; polyanhydrides; polyhydroxy alkonoates such as polyhydroxy valerate, polyhydroxy butyrate, and the like.

The term “primarily” with respect to directional delivery, refers to an amount greater than 50% of the total amount of therapeutic agent provided to a blood vessel.

The term “restenosis” refers to the renarrowing of an artery following an angioplasty procedure which may include stenosis following stent implantation. Restenosis is a wound healing process that reduces the vessel lumen diameter by extracellular matrix deposition, neointimal hyperplasia, and vascular smooth muscle cell proliferation, and which may ultimately result in renarrowing or even reocclusion of the lumen.

The term “anti-restenotic” refers to a drug which interferes with any one or more of the processes of restenosis to reduce the renarrowing of the lumen.

The term “substantially linear release profile” refers to a release profile defined by a plot of the cumulative drug released versus the time during which the release takes place in which the linear least squares fit of such a release profile plot has a correlation coefficient, r² (the square of the correlation coefficient of the least squares regression line), of greater than 0.92 for data time points after the first day of delivery. A substantially linear release profile is clinically significant in that it allows release of a prescribed dosage of drug at a uniform rate over an administration period. This controlled release allows a release system to stay within the toxic/therapeutic window for a particular drug over an extended administration period.

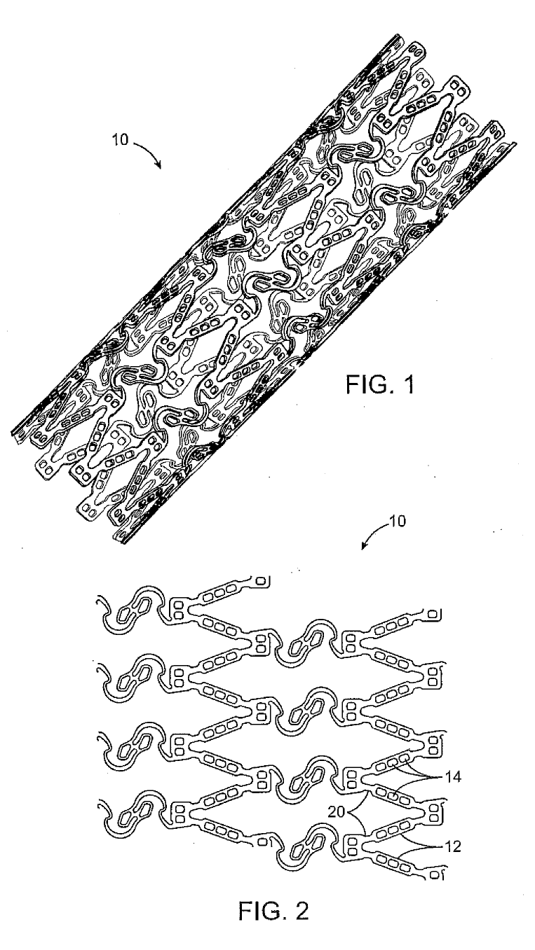

FIG. 1 illustrates one example of an implantable medical device in the form of a stent 10. FIG. 2 is an enlarged flattened view of a portion of the stent of FIG. 1 illustrating one example of a stent structure including struts 12 interconnected by ductile hinges 20. The struts 12 include openings 14 which can be non-deforming through openings containing a therapeutic agent. One example of a stent structure having non-deforming openings is shown in U.S. Pat. No. 6,562,065, which is incorporated herein by reference in its entirety.

The implantable medical devices of the present invention are configured to release at least one therapeutic agent from a matrix affixed to the implantable body. The matrix is formed such that the distribution of the agent in the polymer matrix controls the rate of elution of the agent from the matrix. The release kinetic is also controlled by the selection of the matrix, the concentration of the agent in the matrix, any additives, and any cap or rate controlling deposits.

In one embodiment, the matrix is a polymeric material which acts as a binder or carrier to hold the agent in or on the stent and/or modulate the release of the agent from the stent. The polymeric material can be a bioresorbable or a non-bioresorbable material.

The therapeutic agent containing matrix can be disposed in the stent or on surfaces of the stent in various configurations, including within volumes defined by the stent, such as openings, holes, or concave surfaces, as a reservoir of agent, or arranged in or on all or a portion of surfaces of the stent structure. When the therapeutic agent matrix is disposed within openings in the strut structure of the stent to form a reservoir, the openings may be partially or completely filled with matrix containing the therapeutic agent.

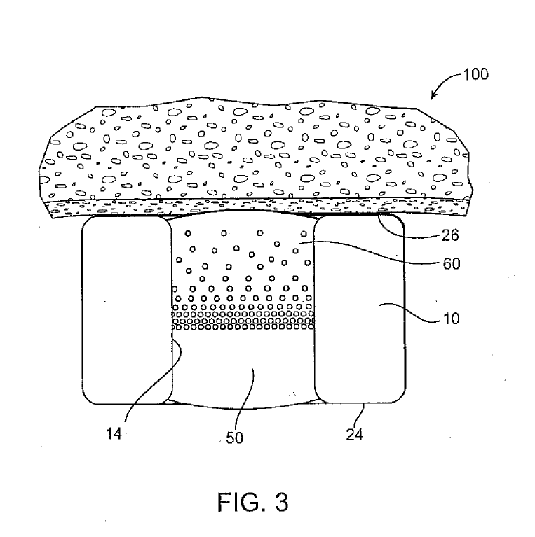

FIG. 3 is a cross section of one strut of the stent 10 and blood vessel 100 illustrating one example of an opening 14 arranged adjacent the vessel wall with a mural surface 26 abutting the vessel wall and a luminal surface 24 opposite the mural surface. The opening 14 of FIG. 3 contains a matrix 60 with a therapeutic agent illustrated by O's in the matrix. The luminal side 24 of the stent opening 14 is provided with a base 50. The base 50 causes the therapeutic agent to be delivered primarily to the mural side 26 of the stent so that it is delivered directly to the artery wall. The base 50 may be formed of a material which also forms the matrix 60 or of a different material. The base 50 can be formed to erode more slowly than the matrix 60 containing the therapeutic agent. This can be achieved by selecting a different molecular weight of the matrix in the base 50, by different processing (i.e., annealing) of the same matrix, or by other means. A thickness of the base 50 can vary from about 5% to about 75%, preferably about 10% to 50%, of the depth of the opening 14.

The matrix 60 and therapeutic agent are arranged in a programmable manner to achieve a desired in vivo release rate and administration period which will be described in further detail below. As can be seen in the example of FIG. 3, the concentration of the therapeutic agent (O's) is highest adjacent the base 50 and transitions to a lower concentration at the mural side 26 of the stent. This configuration and other configurations of concentration gradients within the matrix allow the in vivo release profile to be programmed to match a particular application. In contrast, a uniform agent distribution in the matrix would result in a first order release profile with a large burst followed by a slower release.

The methods by which the drug can be precisely arranged within the matrix in the openings is a stepwise deposition process are further described in U.S. Patent Publications 2005-0010170 and 2004-0073294, both of which are incorporated herein by reference in their entirety.

Numerous other useful arrangements of the matrix and therapeutic agent can be formed to achieve the substantially linear release, increasing release rate, extended release, and substantially complete release described herein. Each of the areas of the matrix may include one or more agents in the same or different proportions from one area to the next. The matrix may be solid, porous, or filled with other drugs or excipients. The agents may be homogeneously disposed or heterogeneously disposed in different areas of the matrix.

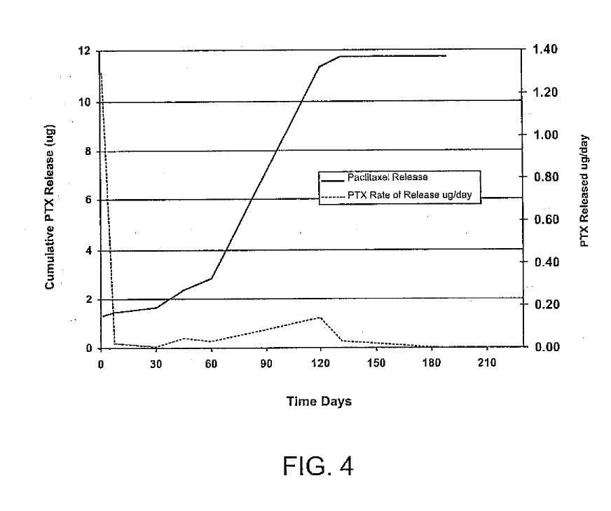

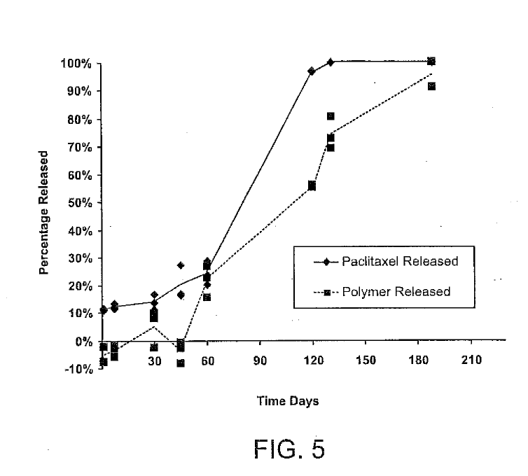

In the example of FIGS. 4 and 5, a stent is cut from a cobalt chromium alloy according to the pattern shown in FIGS. 1 and 2 and paclitaxel is loaded in a PLGA matrix within reservoirs in the stent. The drug and matrix are arranged for directional delivery of the drug to the mural side of the stent. The in vivo drug release rate is programmed by providing different concentrations of drug in different areas of the matrix similar to the concentration gradient shown in FIG. 3. The in vivo drug releases described herein are normalized for a 3.0 mm diameter×16 mm long expanded stent which has almost 500 reservoirs and a total drug volume of about 0.54 mm³.

When the anti-restenotic agent delivered by the method of the invention is paclitaxel, the total amount delivered (and loaded) is preferably between 5 micrograms and 30 micrograms depending on the size of the stent.

The methods of the invention will result in sustained release of substantially all the drug loaded onto the stent as well as the polymer matrix over an administration period which lasts at least 60 days and preferably no longer than 8 months.

FIG. 4 illustrates one example of an in vivo extended paclitaxel release profile from a bioresorbable matrix. The release profile is characterized by a small initial release of drug in the first day, followed by an extended increasing release from day 1 until about 60 to 120 days, followed by a decreasing release until all the drug loaded on the stent is released between about 90 and 180 days. The increasing release rate shown between day 1 and about 90-180 days is different from the releases shown during this time period from coated stents which reach a maximum release rate at a burst in generally the first day and then show a continuously decreasing release rate thereafter.

The increasing in vivo release rate after an initial high release in the first day shown in FIG. 4 more closely matches the delivery of drug to the biological process of restenosis. As shown in FIG. 4 an initial release on day one is followed by a slow release for about days 2-60 and a faster release for about days 60-120. This release curve can be described as having three phases: Phase 1 initial release; Phase 2 release slower than initial release; and Phase 3 release faster than Phase 2 release.

The total drug load on the stents of FIGS. 4 and 5 is between about 10 and about 14 μg normalized for a 3 mm×16 mm stent. The initial release in the first day is about 5-25% of the total amount of paclitaxel loaded on the stent or about 1.5 μg in the first day. The release rate drops to under 0.1 μg per day after day one and continues at this reduced rate for up to about 90 days. A release of between 0.01 μg and 0.2 μg per day continues after day one for at least 60 days and preferably for at least 90 days. A dosage of about 10-14 μg on a 3 mm×16 mm size stent corresponds to about 0.078 μg/mm² of vessel surface area and about 0.732 μg/mm of vessel length. Equivalent dosages are used on stents of other sizes.

The relatively low initial release and slow extended release result in the in vivo release of not more than 40% of the paclitaxel on the stent in the first 30 days after implantation. This is followed by the complete release of the entire dose of paclitaxel loaded on the stent within about 8 months and preferably within about 6 months. A similar in vivo release is also used for other anti-restenotic agents including pimecrolimus and rapamycin which include an initial day one release of up to 25% of the total drug load, a 30 day release of not more than 70% of the total drug load and complete release between 60 days and 8 months.

FIG. 5 illustrates the in vivo release of the paclitaxel from the stent described above compared to the rate that the polymer is resorbed in vivo. The polymer is resorbed at a rate slower than the release of the drug. Therefore, substantially all of the paclitaxel is delivered before the polymer matrix is completely resorbed. In one embodiment the drug is completely delivered about 1-3 months, preferably about 1-2 months, before the polymer is completely resorbed. Preferably, the polymer is completely resorbed between 60 days and 8 months from the date of implantation.

The polymer is resorbed at a rate that is somewhat slower than the release rate of the drug. In the example of FIG. 5, about 10-30% of the polymer is resorbed by about 60 days, about 50-80% of the polymer is resorbed by about 120 days and all the polymer is resorbed between 4 and 7 months. The use of the resorbable polymer which completely disappears from the stent within a period of months allows an administration of antiplatelet drugs to the patient according to current procedures for drug eluting stents to be discontinued after the polymer is completely resorbed and the drug has been released. There is no non-releasable drug or polymer remaining once the stent has been in physiologic conditions for 8 months.

It has been shown in clinical trials that longer in vivo release (greater than 60 days) of the anti-restenotic paclitaxel, such as in the release profiles shown in FIGS. 4 and 5 result in lower in stent neointimal proliferation than the more rapid release of the same dosage. The method of extended in vivo release of anti-restenotic agents increases the therapeutic effectiveness of administration of a given dose of agent and reduces side effects.

While the invention has been described with respect to treatment of restenosis, other therapeutic agents may be delivered at the in vivo release profiles described for treatment of acute myocardial infarction, thrombosis, or for passivation of vulnerable plaque.

Therapeutic Agents

The present invention relates to the in vivo release kinetics involved in delivering anti-restenotic agents including paclitaxel, sirolimus, everolimus, zolarolimus, biolimus, pimecrolimus, cladribine, colchicines, vinca alkaloids, heparin, hinrudin and their derivatives, as well as other cytotoxic or cytostatic, and microtubule stabilizing and microtubule inhibiting agents. These anti-restenotic agents can be delivered alone or in combination.

Although anti-restenotic agents have been primarily described herein, the present invention may also be used to deliver other agents alone or in combination with anti-restenotic agents. Some of the therapeutic agents for use with the present invention which may be transmitted primarily luminally, primarily murally, or both and may be delivered alone or in combination include, but are not limited to, antiproliferatives, antithrombins, immunosuppressants including sirolimus, antilipid agents, anti-inflammatory agents, antineoplastics, antiplatelets, angiogenic agents, anti-angiogenic agents, vitamins, antimitotics, metalloproteinase inhibitors, NO donors, estradiols, anti-sclerosing agents, and vasoactive agents, endothelial growth factors, estrogen, beta blockers, AZ blockers, hormones, statins, insulin growth factors, antioxidants, membrane stabilizing agents, calcium antagonists, retenoid, bivalirudin, phenoxodiol, etoposide, ticlopidine, dipyridamole, and trapidil alone or in combinations with any therapeutic agent mentioned herein. Therapeutic agents also include peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense, oligonucleotides, monoclonal antibodies, platelets, prions, viruses, bacteria, and eukaryotic cells such as endothelial cells, stem cells, ACE inhibitors, monocyte/macrophages or vascular smooth muscle cells to name but a few examples. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the therapeutic layer. Therapeutic agents may also be radioactive isotopes or agents activated by some other form of energy such as light or ultrasonic energy, or by other circulating molecules that can be systemically administered. Therapeutic agents may perform multiple functions including modulating angiogenesis, restenosis, cell proliferation, thrombosis, platelet aggregation, clotting, and vasodilation.

Anti-inflammatories include but are not limited to non-steroidal anti-inflammatories (NSAID), such as aryl acetic acid derivatives, e.g., Diclofenac; aryl propionic acid derivatives, e.g., Naproxen; salicylic acid derivatives, e.g., Diflunisal; and Pimecrolimus. Anti-inflammatories also include glucocoriticoids (steroids) such as dexamethasone, aspirin, prednisolone, and triamcinolone, pirfenidone, meclofenamic acid, tranilast, and nonsteroidal anti-inflammatories. Anti-inflammatories may be used in combination with antiproliferatives to mitigate the reaction of the tissue to the antiproliferative.

The agents can also include anti-lymphocytes; anti-macrophage substances; immunomodulatory agents; cyclooxygenase inhibitors; anti-oxidants; cholesterol-lowering drugs; statins and angiotens in converting enzyne (ACE); fibrinolytics; inhibitors of the intrinsic coagulation cascade; antihyperlipoproteinemics; and anti-platelet agents; anti-metabolites, such as 2-chlorodeoxy adenosine (2-CdA or cladribine); immuno-suppressants including sirolimus, everolimus, tacrolimus, etoposide, and mitoxantrone; anti-leukocytes such as 2-CdA, IL-1 inhibitors, anti-CD116/CD18 monoclonal antibodies, monoclonal antibodies to VCAM or ICAM, zinc protoporphyrin; anti-macrophage substances such as drugs that elevate NO; cell sensitizers to insulin including glitazones; high density lipoproteins (HDL) and derivatives; and synthetic facsimile of HDL, such as lipator, lovestatin, pranastatin, atorvastatin, simvastatin, and statin derivatives; vasodilators, such as adenosine, and dipyridamole; nitric oxide donors; prostaglandins and their derivatives; anti-TNF compounds; hypertension drugs including Beta blockers, ACE inhibitors, and calcium channel blockers; vasoactive substances including vasoactive intestinal polypeptides (VIP); insulin; cell sensitizers to insulin including glitazones, P par agonists, and metformin; protein kinases; antisense oligonucleotides including resten-NG; antiplatelet agents including tirofiban, eptifibatide, and abciximab; cardio protectants including, VIP, insulin, MMP inhibitors, doxycycline, pituitary adenylate cyclase-activating peptide (PACAP), apoA-1 milano, amlodipine, nicorandil, cilostaxone, and thienopyridine; cyclooxygenase inhibitors including COX-1 and COX-2 inhibitors; and petidose inhibitors which increase glycolitic metabolism including omnipatrilat. Other drugs which may be used to treat inflammation include lipid lowering agents, estrogen and progestin, endothelin receptor agonists and interleukin-6 antagonists, and Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAF) dextran; sugar derivatives including D-glucosaminic acid, and D-glucose diethyl mercaptal; synthetic polyethers including polyethylene glycol (PEF and PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

### EXAMPLE

The measurement of in vivo paclitaxel release from a stent can be performed according to the following Example. The in vivo release from other implantable medical devices can be performed in a similar manner by removal of tissue and measurement of total drug load and release kinetics by high pressure liquid chromatography (HPLC).

Stents are implanted in a porcine model and explanted at selected time points by removing the entire artery section. The expanded stents are labeled and frozen. The tissue is removed from the stent by slicing the tissue on the outside of the stent lengthwise, inverting the tissue, and removing the tissue by cutting and turning the tissue inside out. The stent may still be covered by a tough elastic membrane which is then removed by splitting the membrane and peeling it off the stent. For longer time points, there will also be a tub of tissue inside the stent. This tube is separated from the stent with tweezers, turned inside out and pulled out of the stent.

The following is the test procedure for generating the in vivo release curves for paclitaxel in FIGS. 4 and 5. The elution rates of drug from the examples are determined in a standard sink condition experiment.

The total drug load (TDL) of paclitaxel from a stent is determined by extracting all the polymer and drug from the stent in a solvent such as dimethyl sulfoxide (DMSO) or acetonitrile. The amount of paclitaxel in a solution sample is determined by High Pressure Liquid Chromatography (HPLC). The following conditions are used:

Analysis Column: Discovery B10 Wide Pore C5 HPLC Column (150 mm×4.6 mm 5 micron particle)

Mobile phase: Water/Acetonitrile: 56% vol./44% vol.

Flow Rate: 1.0 mL/minute

Temperature: 25° C. ambient

Detection wavelength: 227 nm

Injection volume: 75 μL

Retention time: 14 minutes

The in vivo release kinetic (RK) for paclitaxel from a stent is determined by running the TDL for multiple explanted time points. The TDL for the explanted samples is subtracted from the TDL of an unimplanted stent to determine the amount of paclitaxel released at each of the explanted time points.

The following is the test procedure for generating the in vivo release curve for polymer in FIG. 5. The explanted stents are cleaned of any tissue as described above. The amount of polymer on the stent is determined by thermal analysis thermogravemetric analysis (TGA). The explanted stent is placed on a sensitive balance in a controlled atmosphere furnace where the furnace temperature is slowly increased from 25 to 440° C. at a rate of 5° C. per minute. Different constituents in the sample vaporize at different temperatures beginning with residual solvent followed by polymer plus drug. The temperatures of vaporization of polymer and drug are sufficiently close that the weight of polymer and drug together is determined. The amount of polymer is calculated as the difference between the weight loss measured by thermogravimetric analysis minus the weight of drug measured according to the paclitaxel TDL procedure. This procedure is then repeated for the multiple explanted time points to determine the in vivo release curve for polymer.

While the invention has been described in detail with reference to the preferred embodiments thereof, it will be apparent to one skilled in the art that various changes and modifications can be made and equivalents employed, without departing from the present invention.

What is claimed is:

1. A method of reducing restenosis comprising:
providing a drug delivery stent having a dosage of paclitaxel for delivery to an artery, the dosage arranged such that substantially all the paclitaxel is releasable from the stent upon implantation of the stent in the artery;
implanting the stent within an artery of a patient; and
delivering paclitaxel from the stent in vivo over an administration period beginning on the date of implantation and ending between 60 days and 8 months after implantation, wherein after the administration period no paclitaxel remains on the stent.

2. The method of claim 1, wherein the administration period ends between about 90 and 180 days from the date of implantation.

3. The method of claim 1, wherein the release profile of the paclitaxel after day one is substantially linear.

4. The method of claim 1, wherein the amount of paclitaxel released per day after day one is about 0.01 to about 0.2 μg per day delivered from a 3 mm by 16 mm expanded stent, and equivalent dosages are delivered from stents of other sizes.

5. The method of claim 1, wherein the paclitaxel is deposited in openings in the stent.

6. The method of claim 1, wherein the paclitaxel is contained in a bioresorbable matrix.

7. The method of claim 1, wherein the paclitaxel is contained in a polymer matrix.

8. The method of claim 7, wherein the polymer matrix is completely resorbed between 60 days and 8 months from the date of implantation.

9. The method of claim 8, wherein the step of delivering paclitaxel further comprises delivering substantially all the paclitaxel from the stent before the polymer matrix is completely resorbed.

10. The method of claim 1, wherein the paclitaxel is delivered primarily murally from the stent.

11. The method of claim 1, wherein the step of delivering paclitaxel further comprises delivering 5-25% of the total amount of paclitaxel loaded into the stent in the first day.

12. The method of claim 1, wherein the step of delivering paclitaxel further comprises delivering paclitaxel at an increasing release rate between days 1 and 60 after implantation.

13. The method of claim 1, wherein the step of delivering paclitaxel further comprises delivering substantially all of the paclitaxel loaded on the stent in no longer than 180 days.

14. The method of claim 1, wherein the step of delivering paclitaxel delivers not more than 40% of the paclitaxel in the first 30 days.

15. A method of reducing restenosis comprising:
providing a drug delivery stent having a dosage of antirestenotic drug for delivery to an artery, the dosage arranged such that substantially all the drug is releasable from the stent upon implantation of the stent in the artery;
implanting the stent within an artery of a patient; and
delivering the drug from the stent in vivo over an administration period beginning on the date of implantation and ending between 60 days and 8 months after implantation, wherein after the administration period no drug remains on the stent.

16. The method of claim 15, wherein the administration period ends between about 90 and 180 days from the date of implantation.

17. The method of claim 15, wherein the release profile of the drug after day one is substantially linear.

18. The method of claim 15, wherein the amount of drug released per day after day one is about 0.01 to about 0.2 μg per day delivered from a 3 mm by 16 mm expanded stent, and equivalent dosages are delivered from stents of other sizes.

19. The method of claim 15, wherein the drug is deposited in openings in the stent.

20. The method of claim 15, wherein the drug is contained in a bioresorbable matrix.

21. The method of claim 15, wherein the drug is contained in a polymer matrix.

22. The method of claim 21, wherein the polymer matrix is completely resorbed between 60 days and 8 months from the date of implantation.

23. The method of claim 22, wherein the step of delivering drug further comprises delivering substantially all the drug from the stent before the polymer matrix is completely resorbed.

24. The method of claim 15, wherein the drug is delivered primarily murally from the stent.

25. The method of claim 15, wherein the step of delivering drug further comprises delivering 5-25% of the total amount of drug loaded into the stent in the first day.

26. The method of claim 15, wherein the step of delivering drug further comprises delivering drug at an increasing release rate between days 1 and 60 after implantation.

27. The method of claim 15, wherein the step of delivering drug further comprises delivering substantially all of the drug loaded on the stent in no longer than 180 days.

28. A stent for reducing restenosis comprising:
a drug delivery stent having initial unexpanded diameter for insertion of the stent into a coronary artery and an expanded diameter for implantation within a coronary artery, the stent having a dosage of paclitaxel for delivery to an artery, the dosage arranged such that substantially all the paclitaxel is releasable from the stent upon implantation of the stent in the artery, wherein the dosage of paclitaxel is arranged to be released over an in vivo administration period beginning on the date of implantation and ending between 60 days and 8 months after implantation, and wherein after the administration period no drug remains on the stent.

29. The stent of claim 28, wherein the administration period ends between about 90 and 180 days from the date of implantation.

30. The stent of claim 28, wherein the release rate of the paclitaxel after day one is substantially linear.

31. The stent of claim 28, wherein the amount of paclitaxel released per day after day one is about 0.01 to about 0.2 μg per day delivered from a 3 mm by 16 mm expanded stent, and equivalent dosages are delivered from stents of other sizes.

32. The stent of claim 28, wherein the paclitaxel is deposited in openings in the stent.

33. The stent of claim 28, wherein the paclitaxel is contained in a bioresorbable matrix.

34. The stent of claim 28, wherein the paelitaxel is contained in a polymer matrix.

35. The stent of claim 34, wherein the polymer matrix is completely resorbed between 60 days and 8 months from the date of implantation.

36. The stent of claim 35, wherein the polymer matrix is selected to delivery substantially all the paclitaxel from the stent before the polymer matrix is completely resorbed.

37. The stent of claim 28, wherein the paclitaxel is arranged to be delivered primarily murally from the stent.

38. The stent of claim 28, wherein the paclitaxel is affixed to the stent such that 5-25% of the total amount of paclitaxel loaded into the stent is delivered in the first day.

39. The stent of claim 28, wherein the paclitaxel is loaded for delivery at an increasing release rate between days 1 and 60 after implantation.

40. A method of reducing restenosis comprising:
providing a drug delivery stent having a dosage of antirestenotic drug for delivery to an artery;
implanting the stent within an artery of a patient; and
delivering drug from the stent in vivo over an administration period beginning on the date of implantation and ending within 6 months after implantation, wherein not more than 40% of the drug is delivered in the first 30 days and after the administration period no drug remains on the stent.

41. The method of claim 40, wherein the drug is deposited in openings in the stent.

42. The method of claim 40, wherein the drug is contained in a bioresorbable matrix.

43. The method of claim 40, wherein the drug is contained in a polymer matrix.

44. The method of claim 40, wherein the drug is delivered primarily murally from the stent.
